(19) United States
(12) Patent Application Publication
(10) Pub. No.: US 2020/0040348 A1
(43) Pub. Date: Feb. 6, 2020

(54) CONTROL OF VIRAL AND BACTERIAL INFECTION BY ANTIMICROBIAL PEPTIDES RETROCYCLIN AND/OR PROTEGRIN EXPRESSED IN CHLOROPLASTS

(71) Applicant: THE TRUSTEES OF THE UNIVERSITY OF PENNSYLVANIA, Philadelphia, PA (US)

(72) Inventor: Henry Daniell, Media, PA (US)

(21) Appl. No.: 16/504,792

(22) Filed: Jul. 8, 2019

Related U.S. Application Data
(63) Continuation of application No. 13/101,389, filed on May 5, 2011, now abandoned.
(60) Provisional application No. 61/331,794, filed on May 5, 2010.

(51) Int. Cl. C12N 15/82; C07K 14/46

(52) U.S. Cl. CPC C12N 15/8214; C07K 14/46; A61K 38/00; C12N 15/8281; C12N 15/8257; C12N 15/8283

(57) ABSTRACT

Disclosed herein are antimicrobial compositions containing one or more antimicrobial peptides having been expressed in chloroplasts. Exemplified herein are the expression and use of retrocylin and protegrin. Disclosed herein are methods of engineering chloroplasts to express such antimicrobial peptides such that they are properly processed and active. Plants containing such chloroplasts are disclosed as well. The chloroplast expressed peptides are useful to delay, prevent or treat viral and bacterial infections.

### RELATED APPLICATIONS

This application is a continuation of U.S. patent application Ser. No. 13/101,389, filed May 5, 2011, which claims benefit of U.S. Provisional Application No. 61/331,794, filed May 5, 2010. The entire disclosure of each the aforesaid application is incorporated by reference in the present application.

### GOVERNMENT SUPPORT

This work was supported by NIH R01 GM 63879 and USDA 3611-21000-021-02S grants. The U.S. government has certain rights in this invention.

### INCORPORATION-BY-REFERENCE OF MATERIAL SUBMITTED IN ELECTRONIC FORM

Applicant hereby incorporates by reference the Sequence Listing material filed in electronic form herewith. This file is labeled “UPNK_5935US01_SeqListing_ST25.txt”, dated Oct. 28, 2019 and is 11,401 bytes in size.

### BACKGROUND OF THE INVENTION

Antimicrobial peptides are evolutionarily conserved components of the innate immune response and are found in different organisms, including bacteria, vertebrates, invertebrates and plants (Boman, H. G., (1997) Peptide antibiotics and their role in annate immunity. Annu. Rev. Immunol. 13, 61-92; Broekaert, W. F., et al. (1997) Antimicrobial Peptides from Plants. Crit. Rev. Plant. Sci. 16, 297-323; Hancock and Chapple, (1999) Peptide Antibiotics. Antimicrob. Agents Chemother. 43, 1317-1323; Nicolas and Mor, (1995) Peptides as weapons against microorganisms in the chemical defense system of vertebrates. Annu. Rev. Microbiol. 49, 277-304). Antimicrobial peptides are also called peptide antibiotics. When compared with conventional antibiotics, development of resistance is less likely with antimicrobial peptides. Many bacteria species remain sensitive to antimicrobial peptides after a long time of evolution (Nizet, V. (2006) Antimicrobial peptide resistance mechanisms of human bacterial pathogens. Curr. Issues Mol. Biol. 8, 11-26; Yeaman, M. R. and Yount, N. Y. (2003) Mechanisms of Antimicrobial Peptide Action and Resistance. Pharmacol. Rev. 55, 27-55). Adaptive immune systems can remember the pathogen and elicit a much faster and stronger immune response against that pathogen at subsequent encounters (Boman, H. G. (1995) Peptide antibiotics and their role in innate immunity. Annu. Rev. Immunol. 13, 61-92). Without such specificity and memory, antimicrobial peptides evolved a different mechanism against pathogen infections. Most antimicrobial peptides are efficient against a broad-spectrum of pathogens rather than specific against one pathogen, which makes them especially suitable for use against local and systematic infections (Bals, R. (2000) Epithelial antimicrobial peptides in host defense against infection. Respir. Res. 1, 141-150; Schaller-Bals et al., (2002) Increased levels of antimicrobial peptides in tracheal aspirates of newborn infants during infection. Am. J Respir. Crit Care Med. 165, 992-995). Other than the antimicrobial activities, some antimicrobial peptides are shown to have immunomodulatory activities. Some studies show that antimicrobial peptides like defensins are likely to play a role in recruiting effector T cells to inflammatory sites, thereby contributing to the effector phase of adaptive immunity (Yang et al., (2001) The role of mammalian antimicrobial peptides and proteins in awakening of innate host defenses and adaptive immunity. Cell Mol. Life Sci. 58, 978-989). These intriguing characteristics of antimicrobial peptides facilitate development of novel antibiotics. However, the high cost of production of antimicrobial peptides and lack of suitable expression systems could be potential barriers for their development and clinical studies.

The chloroplast, as a bioreactor, is able to express foreign proteins at high levels because of its high copy number. When a transgene is integrated into the inverted repeat region of the chloroplast genome, up to 20,000 copies of the transgene per cell could be expressed. Several therapeutic proteins have been expressed in chloroplasts, including human blood proteins somatotropin (Staub et al., (2000) High-yield production of a human therapeutic protein in tobacco chloroplasts. Nat. Biotechnol. 18, 333-338), insulin like growth factor (Daniell et al., (2005) Chloroplast-derived vaccine antigens and other therapeutic proteins. Vaccine 23, 1779-1783), proinsulin (Ruhlman et al., (2007) The role of heterologous chloroplast sequence elements in transgene integration and expression. Plant Physiol., 152, 2088-2104), IFN-α2b (Arlen et al., (2005) Field production and functional evaluation of chloroplast-derived interferon-alpha2b. Plant Biotechnol. J. 5, 511-525), serum albumin (Fernandez-San et al., A chloroplast transgenic approach to hyper-express and purify Human Serum Albumin, a protein highly susceptible to proteolytic degradation. Plant Biotechnol. J. 1, 71-79, 2003), IFN-γ (Leelavathi, S. and Reddy, V. S., Chloroplast expression of His-tagged GUS-fusions: a general strategy to overproduce and purify foreign proteins using transplastomic plants as bioreactors. Molecular Breeding 11, 49-58, 2003), cardiotrophin-1 (Farran et al., High-density seedling expression system for the production of bioactive human cardiotrophin-1, a potential therapeutic cytokine, in transgenic tobacco chloroplasts. Plant Biotechnol. J. 6, 516-527, 2008), alpha1-antitrypsin (Nadai et al. High-level expression of active human alpha1-antitrypsin in transgenic tobacco chloroplasts. Transgenic Res. 18, 173-183-2009) and glutamic acid decarboxylase (Wang et al., A novel expression platform for the production of diabetes-associated autoantigen human glutamic acid decarboxylase (hGAD65). BMC. Biotechnol. 8, 87-90, 2008). In addition, several vaccine antigens have been expressed in chloroplasts against several bacterial pathogens including cholera toxin B subunit (Daniell et al., Expression of the native cholera toxin B subunit gene and assembly as functional oligomers in transgenic tobacco chloroplasts. J. Mol. Biol. 311, 1001-1009, 2001), tetanus toxin (Tregoning et al., Expression of tetanus toxin Fragment C in tobacco chloroplasts. Nucleic Acids Res. 31, 1174-1179, 2003), anthrax protective antigen (Koya et al., Plant-based vaccine: mice immunized with chloroplast-derived anthrax protective antigen survive anthrax lethal toxin challenge. Infect. Immun. 73, 8266-8274, 2005; Watson et al., Expression of Bacillus anthracis protective antigen in transgenic chloroplasts of tobacco, a non-food/feed crop. Vaccine 22, 4374-4384, 2004), plague F1-V fusion antigen (Arlen et al., Effective plague vaccination via oral delivery of plant cells expressing F1-V antigens in chloroplasts. Infect. Immun. 76, 3640-3650, 2008), outer surface lipoprotein A (OspA) for Lyme disease (Glenz et al., Production of a recombinant bacteriallipoprotein in higher plant chloroplasts. Nat. Biotechnol. 24, 76-77, 2006) and their functionality have been evaluated in cell culture systems or animal models after pathogen or toxin challenges. Antigens produced against protozoan pathogens were immunogenic against amoeba (Chebolu and Daniell, 2007) or effective against the malarial parasite (Davoodi-Semiromi et al., The green vaccine: A global strategy to combat infectious and autoimmune diseases. Hum. Vaccin. 5, 488-493, 2009). Although several viral antigens have been expressed in chloroplasts, neutralizing antibodies were shown only against human papillomavirus (Fernandez-San et al., Human papillomavirus L1 protein expressed in tobacco chloroplasts self-assembles into virus-like particles that are highly immunogenic. Plant Biotechnol. J. 6, 427-441, 2008) and canine parvovirus 2L21 peptide (Molina et al., High-yield expression of a viral peptide animal vaccine in transgenic tobacco chloroplasts. Plant Biotechnol. J. 2, 141-153, 2004). Other proteins expressed in chloroplasts include bovine mammary-associated serum amyloid (Manuell et al., Robust expression of a bioactive mammalian protein in Chlamydomonas chloroplast. Plant Biotechnol. J. 5, 402-412, 2007), aprotinin (Tissot et al., Translocation of aprotinin, a therapeutic protease inhibitor, into the thylakoid lumen of genetically engineered tobacco chloroplasts. Plant Biotechnol. J. 6, 309-320, 2008) and monoclonal large single-chain (lsc) antibody against glycoprotein D of the herpes simplex virus (Mayfield et al., Expression and assembly of a fully active antibody in algae. Proc. Natl. Acad. Sci. U.S.A. 100, 438-442, 2003). The expression levels of these proteins are mostly 2-20% of TSP, but could be even higher than RuBisCo (Oey et al., Exhaustion of the chloroplast protein synthesis capacity by massive expression of a highly stable protein antibiotic. Plant J. 57, 436-445, 2009; Ruhlman et al., 2010). Other advantages of chloroplast transformation include multigene engineering, transgene containment, lack of position effect, gene silencing and maternal inheritance (Daniell et al., Plant-made vaccine antigens and biopharmaceuticals. Trends Plant Sci. 14, 669-679, 2009).

Retrocyclin is a cyclic octadecapeptide, which is artificially synthesized based on a human pseudogene that is homologous to rhesus monkey circular minidefensins. Retrocyclin contains six cysteines, and has largely β-sheet structure that is stabilized by three intramolecular disulfide bonds. Structure-function studies indicate that the cyclic backbone, intramolecular tri-disulfide ladder, and arginine residues of retrocyclin contributed substantially to its protective effects (Jenssen et al., Peptide Antimicrobial Agents. Clin. Microbiol. Rev. 19, 491-511, 2006; Trabi et al., 2001). Retrocyclin peptides are small antimicrobial agents with potent activity against bacteria and viruses, especially against HIV retrovirus or sexually-transmitted bacteria. Previous studies have shown that RC-101 and other retrocyclins can protect human CD4⁺ cells from infection by T- and M-tropic strains of HIV-1 in vitro (Cole et al., Retrocyclin: A primate peptide that protects cells from infection by T- and M-tropic strains of HIV-1. Proceedings of the National Academy of Sciences 99, 1813-1818, 2002) and prevent HIV-1 infection in an organ-like construct of human cervicovaginal tissue (Cole et al., The retrocyclin analogue RC-101 prevents human immunodeficiency virus type 1 infection of a model human cervicovaginal tissue construct. Immunology 121, 140-145, 2007). The ability of RC-101 to prevent HIV-1 infection and retain full activity in the presence of vaginal fluid makes it a good candidate for topical microbicide to prevent sexual transmission of HIV-1.

Protegrin-1 (PG1) belongs to the protegrin family, which is discovered in porcine leukocytes (Kokryakov et al., Protegrins: leukocyte antimicrobial peptides that combine features of corticostatic defensins and tachyplesins. FEBS Letters 327, 231-236, 1993). PG1 is a cysteine-rich, 18-residue β-sheet peptide. It has a high content of arginine, an amidated C-terminus, and four conserved cysteines at positions 6, 8, 13, and 15 which would form two disulfide bonds. The antimicrobial activity of PG1 is strongly related to the stability of β-hairpin confirmation and the β-hairpin confirmation of PG1 is stabilized by the two disulfide bonds. Removal of both disulfide bonds would result in substantial reduction of PG1's activity (Chen et al., Development of protegrins for the treatment and prevention of oral mucositis: structure-activity relationships of synthetic protegrin analogues. Biopolymers. 55, 88-98, 2000; Harwig et al., Intramolecular disulfide bonds enhance the antimicrobial and lytic activities of protegrins at physiological sodium chloride concentrations. Eur. J Biochem. 240, 352-357, 1996). Therefore, the disulfide bridges are very important to the activity of PG1. It was shown that PG1 had potent antimicrobial activity against a broad spectrum of microorganisms, including bacteria, fungi and yeasts (Kokryakov et al., Protegrins: leukocyte antimicrobian peptides that combine features of corticostatic defensins and tachyplesins. FEBS Letters 327, 231-236, 1993; Steinberg et al., Protegrin-1: a broad-spectrum, rapidly microbicidal peptide with in vivo activity. Antimicrob. Agents Chemother. 41, 1738-1742, 1997). Chlamydia trachomatis and Neisseria gonorrhoeae are two kinds of pathogenic bacteria which can cause sexually transmitted diseases (STDs) in humans. Two previous studies that compare the efficiency of PG1 with human neutrophil defensins demonstrated that PG1 is more potent than human neutrophil defensins in inactivating Chlamydia trachomatis and Neisseria gonorrhoeae (Qu et al., Susceptibility of Neisseria gonorrhoeae to protegrins. Infect. Immun. 64, 1240-1245, 1996; Yasin et al., Susceptibility of Chlamydia trachomatis to protegrins and defensins. Infect. Immun. 64, 709-713, 1996). In a previous study, the inventors expressed the antimicrobial peptide MSI-99, an analog of magainin 2, via the chloroplast genome to obtain high levels of protection against bacterial and fungal pathogens (DeGray et al., Expression of an antimicrobial peptide via the chloroplast genome to control phytopathogenic bacteria and fungi. Plant Physiol 127, 852-862, 2001). Recently, a proteinaceous antibiotic, PlyGBS lysin was also expressed in the chloroplast and it was shown that the protein synthesis capacity of the chloroplast was exhausted by the massive production of the foreign protein (Oey et al., Exhaustion of the chloroplast protein synthesis capacity by massive expression of a highly stable protein antibiotic. Plant J. 57, 436-445, 2009). However, antimicrobial peptides containing multiple intramolecular disulfide bonds have not yet been expressed in chloroplasts.

### SUMMARY OF THE INVENTION

In light of the problems found in the prior art, it has been discovered that retrocylin and/or protegrin can be expressed and properly processed in chloroplasts. Chloroplast expressed retrocylin and protegrin possesses an unexpectedly high antimicrobial activity and should be able to inactivate most bacterial and viral pathogens. In particular, the evidence shows that such peptides are especially effective against bacteria and viruses that cause STDs. In one embodiment, the subject invention includes a method of treating, preventing or delaying the onset of a viral or bacterial infection, comprising administering to a subject a composition comprising a therapeutically effective amount of an antimicrobial peptide expressed in and obtained from a chloroplast.

In another embodiment, a stable plastid transformation and expression vector is provided. The vector comprises an expression cassette comprising, as operably linked components in the 5′ to the 3′ direction of translation, a promoter operative in said plastid, a selectable marker sequence, a heterologous polynucleotide sequence coding for a polypeptide comprising at least 70, 80, 90, 92, 93, 94, 95, 96, 97, 98 or 99% identity to a retrocyclin or protegrin, transcription termination functional in said plastid, and flanking each side of the expression cassette. The vector further comprises flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome.

In a further embodiment, a stably transformed plant which comprises plastid stably transformed with the aforementioned vector or the progeny thereof, including seeds is provided. In yet a further embodiment, a process for producing a retrocyclin or protegrin polypeptide is provided. The process comprising: integrating a plastid transformation vector according to the vector embodiment above into the plastid genome of a plant cell; and growing said plant cell to thereby express said retrocyclin or protegrin.

In still a further embodiment is a plastid genome transformed to contain a retrocyclin or protegrin polynucleotide configured so as to express retrocyclin or protegrin.

### BRIEF DESCRIPTION OF THE DRAWINGS

FIG. 1A-FIG. 1E shows a schematic representation of chloroplast vectors. FIG. 1A demonstrates the native chloroplast genome showing both homologous recombination sites (trnI and trnA) and the restriction enzyme sites used for Southern blot analysis. FIG. 1B illustrates the pLD-His₆-GFP-Furin-PG1 vector map with the primer annealing sites. FIG. 1C illustrates the pLD-GFP-His₆-Factor Xa-RC101 vector map, the primer annealing sites are the same as shown on the PG1 vector map. FIG. 1D shows the nucleotide sequence of GFP-6×His-Factor Xa-RC101 and the schematic representation of disulfide bonds in RC101. FIG. 1E provides the nucleotide sequence of 6×His-GFP-Furin-PG1 and the schematic representation of disulfide bonds in PG1.

FIG. 2A-FIG. 2C shows a PCR and Southern blot analysis is shown, which is performed in order to investigate trangene integration and homoplasmy. FIG. 2A is a PCR analysis of the untransformed and transplastomic lines using the primer pair 3P/3M. Lanes 1-3 are RC-101 transplastomic lines, and lanes 4-6 are PG1 transplastomic lines. FIG. 2B demonstrates a PCR analysis of the untransformed and transplastomic lines using the primer pair 5P/2M. Lanes 1-3 are RC-101 transplastomic lines, and lanes 4-6 are PG1 transplastomic lines. FIG. 2C shows a Southern blot hybridized with the flanking sequence trnI-trnA probe to investigate the homoplasmy of RC101 and PG1 transplastomic lines. Lanes 1-2 are DNA samples from RC101 transplastomic plants, and lanes 3-4 are PG1 transplastomic plants. M is 1 kbp DNA plus ladder, and WT is untransformed tobacco.

FIG. 3A-FIG. 3D demonstrates protease cleavage of the fusion proteins by immunoblot and quantification of expression by densitometric analysis. FIG. 3A is an immunoblot analysis of RC101-GFP and PG1-GFP expression and cleavage. Lane 1 is untransformed protein extract, 10 μg, lane 2 is Precision Plus protein marker, 5 μg, lane 3 is RC101-GFP transplastomic line protein extract, 3 μg, and lane 4 is RC101-GFP protein extract digested by Factor Xa protease, 3 μg. Lane 5 is PG1-GFP protein extract, 6 μg, lane 6 is PG1-GFP protein extract digested by furin protease, 6 μg, and lane 7 is GFP standard, 100 ng. FIG. 3B is the native polyacrylamide gel electrophoresis of RC101-GFP and PG1-GFP protein extracts. Lanes 1-3 are GFP standard (150, 300, 600 ng), lane 4 is untransformed plant extract, 10 μg, lanes 5-6 are RC101 transplastomic extracts (6, 8 μg), and lanes 7-8 are PG1 transplastomic extracts (6, 8 μg). FIG. 3C is the GFP standard curve based on the IDVs of 150, 300 and 600 ng of GFP standard. FIG. 3D shows the estimation of RC101-GFP and PG1-GFP expression levels in transplastomic plants.

FIG. 4A-4B provides dot blot analysis and silver staining that was performed to investigate expression of RC101 and PG1. FIG. 4A shows dot blot analysis of RC101 before and after cleavage. Indicated amount of RC101 was used as standards. Uncut, RC101-GFP without Factor Xa cleavage; Cut, RC101-GFP after Factor Xa cleavage. IG. 4B shows silver stained gel of plant extracts before or after furin cleavage of PG1-GFP protein. Lane 1 is Marker 12 (invitrogen), lane 2 is untransformed plant protein extract, 40 μg, lane 3 is PG1-GFP protein extract without furin digestion, 40 μg, and lane 4 is PG1-GFP protein extract digested by furin protease, 40 μg.

FIG. 5A-FIG. 5B provides the result of confocal microscopy of RC101-GFP and PG1-GFP transplastomic plants. The left panels shows chloroplasts from RC101-GFP (FIG. 5A) or PG1-GFP (FIG. 5B) transplastomic lines (bars=20 μm). The right panels showed four times higher magnification of the boxed regions (bars=5 μm).

FIG. 6 shows that purified RC101-GFP and PG1-GFP fusion proteins were separated on native PAGE and observed by Coomassie staining or fluorescence under UV light. PG1-GFP was purified by affinity chromatography and RC101-GFP was purified by both affinity chromatography and organic extraction method. Samples were loaded in duplicate. M, Precision Plus protein marker, 5 μg; St, GFP standard, 500 ng. The same gel was observed under UV light (bottom) or stained by Coomassie staining (top). The yield of GFP-RC 101 was 5 μg/g leaf by affinity chromatography and 53 μg/g leaf by organic extraction; GFP-PG1 yield was 8 5 μg/g leaf. by affinity chromatography purification.

FIG. 7 shows in planta antimicrobial bioassays performed to investigate functionality of RC101 and PG1 expressed in chloroplasts. Twenty μl of the 10⁸, 10⁶, 10⁴ and 10² cells from an overnight culture of E. carotovora were injected into leaves of (FIG. 7A) RC101, (FIG. 7E) PG-1 transplastomic, and (FIG. 7B, FIG. 7F) untransformed (UT) plants using a syringe with a precision glide needle. Five- to 7-mm areas of (FIG. 7C) RC101, (FIG. 7G) PG-1 and (FIG. 7D, FIG. 7H) untransformed leaves were scraped with fine-grain sandpaper. Twenty μl 10⁸, 10⁶, 10⁴ and 10² cells of Erwinia were inoculated to each prepared area. Photos were taken 5 days after inoculation.

FIG. 8 provides the bacterial density in the PG1, RC101 and untransformed (UT) plants inoculated with E. carotovora. FIG. 8A shows bacterial density in RC101 and untransformed leaves, whereas FIG. 8b provides the bacterial density in PG1 and untransformed leaves. The bacterial density is shown in plants on 0, 1 and 3 days after inoculation. All values represent means of 6 replications with standard deviations shown as error bars.

FIG. 9 demonstrates the response of untransformed and RC101/PG1 transplastomic plants to TMV. FIG. 9A shows a TMV inoculated leaf from an untransformed plant; FIG. 9B shows a TMV inoculated leaf from a transplastomic PG1 plant, and FIG. 9C shows a TMV inoculated leaf from a transplastomic RC101 plant. The pictures were taken on 20 days after inoculation.

### DETAILED DESCRIPTION

Retrocyclin peptides are used, in particular, for their potent activity against bacteria and virus, especially against HIV retrovirus or sexually-transmitted bacteria. As a small antimicrobial agent, retrocyclin (RC-101) has an ability to prevent HIV-1 infection and also retains full activity in the presence of vaginal fluid, making it a good candidate for topical microbicide in the prevention of sexual transmission of HIV-1.

Protegrin (PG1) has potent antimicrobial activity against bacteria, fungi, yeasts, and other microorganisms. However, PG1 has shown greater anti-bacterial than anti-viral activity. PG1 has been particularly effective in inactivating two specific types of pathogenic bacteria, namely Chlamydia trachomatis and Neisseria gonorrhoeae. Therefore, the inventors have discovered that the combination of RC-101 and PG1 would be especially effective against both bacteria and viruses that cause STDs.

It has been identified that expression of functional disulfide-bonded antimicrobial peptides in chloroplasts. The inventors have noted that chloroplasts have already been shown in previous studies to be fully functional in expressing biologically active, disulfide-bonded therapeutic proteins, such as human somatotropin (Staub et al., High-yield production of a human therapeutic protein in tobacco chloroplasts. Nat. Biotechnol. 18, 333-338, 2000), cholera toxin B (Daniell et al., Expression of the native cholera toxin B subunit gene and assembly as functional oligomers in transgenic tobacco chloroplasts. J. Mol. Biol. 311, 1001-1009, 2001), human interferon-α2b (Arlen et al., Field production and functional evaluation of chloroplast-derived interferon-alpha2b. Plant Biotechnol. J. 5, 511-525, 2007) and alkaline phosphatases (Bally et al., Both the stroma and thylakoid lumen of tobacco chloroplasts are competent for the formation of disulphide bonds in recombinant proteins. Plant Biotechnol. J. 6, 46-61, 2008). However, chemical synthesis is very expensive, and production of these proteins is challenging. Because of the high cost associated with chemical synthesis and inability of cell culture or microbial systems to produce these proteins, the inventors have found the expression of RC101 or PG1 antimicrobial peptides in chloroplasts to be an ideal solution for large scale economic production.

Therefore, in an embodiment of the subject invention, a stable plastid transformation and expression vector is provided. The vector includes an expression cassette, including, as operably linked components in the 5′ to the 3′ direction of translation, a promoter operative in said plastid, a selectable marker sequence, and a heterologous polynucleotide sequence coding for a polypeptide comprising at least 70, 80, 90, 92, 93, 94, 95, 96, 97, 98 or 99% identity to a retrocyclin or protegrin. The vector also includes transcription termination functional in said plastid, and flanking each side of the expression cassette. The vector further includes flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome. In a particular embodiment, the vector is provided wherein the selectable marker sequence is an antibiotic-free selectable marker.

In a more particular embodiment, the plastid is selected from the group consisting of chloroplasts, chromoplasts, amyloplasts, proplastids, leucoplasts and etioplasts.

Chloroplasts, organelles found in plant cells and other eukaryotic organisms, conduct photosynthesis. Chloroplasts capture light energy to conserve free energy in the form of ATP and reduce NADP to NADPH through photosynthesis. Campbell, Neil A.; Brad Williamson; Robin J. Heyden (2006). Biology: Exploring Life. Boston, Mass.: Pearson Prentice Hall. ISBN 978-0-13-250882-7. Chromoplasts are, like all other plastids (including chloroplasts and leucoplasts) organelles found in specific photosynthetic and eukaryotic species. They are the plastids responsible for pigment synthesis and storage.

Amyloplasts are non-pigmented organelles found in some plant cells. They are responsible for the synthesis and storage of starch granules, through the polymerization of glucose. Wise, Robert (2007) The Diversity of Plastid Form and Function. Springer. springerlink.com/index/qp032630631337u6.pdf. Proplastids are undifferentiated plastids. All plastids are derived from proplastids, they are present in the meristematic regions of the plant. Proplastids and young chloroplasts commonly divide, but more mature chloroplasts also have this capacity. Plastids in plants differentiate into several forms, proplastids may develop into any of: chloroplasts, chromoplasts, gerontoplasts, leucoplasts, amyloplasts, elaioplasts, or proteinoplasts. Leucoplasts can be differentiated from other plastids because they are non-pigmented. Amyloplasts are used for starch storage and detecting gravity in the plant, and elaioplasts are used for storing fat. The function of proteinoplasts is for storage and modification of protein in the plant. Proteinoplasts contain crystalline bodies of protein and can be the sites of enzyme activity involving those proteins.

In another embodiment, a stably transformed plant which includes plastid stably transformed with a vector including an expression cassette, including, as operably linked components in the 5′ to the 3′ direction of translation, a promoter operative in said plastid, a selectable marker sequence, and a heterologous polynucleotide sequence coding for a polypeptide comprising at least 70, 80, 90, 92, 93, 94, 95, 96, 97, 98 or 99% identity to a retrocyclin or protegrin. The vector also includes transcription termination functional in said plastid, and flanking each side of the expression cassette. The vector further includes flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome. The stably transformed plant comprising plastid stably transformed with this vector, or the progeny thereof, including seeds. All of the chloroplasts of the stably transformed plant may be uniformly transformed. The stably transformed plant may be either a monocotyledonous or dicotyledonous plant. The plant may be maize, rice, grass, rye, barley, oat, wheat, soybean, peanut, grape, potato, sweet potato, pea, canola, tobacco, tomato, or cotton. The stably transformed plant may be edible for mammals and humans.

In a further embodiment, a method of treating, preventing or delaying the onset of a viral or bacterial infection is provided. The method includes administering to a subject a composition including a therapeutically effective amount of an antimicrobial peptide expressed in and obtained from a chloroplast. As used herein, the term “subject” refers to an animal, preferably a mammal such as a non-primate (e.g., cows, pigs, horses, cats, dogs, rats etc.) and a primate (e.g., monkey and human).

In a more particular embodiment, a therapeutically effective amount of the composition is adminstered topically, intramuscularly, intravaginally, transdermally, orally, or intravenously.

The amount that is denoted as a “therapeutically effective amount,” as used herein, depends on the subject and the circumstances. The amount administered to an animal, particularly a human, in accordance with the present invention should be sufficient to effect the desired response in the animal over a reasonable time frame. One skilled in the art will recognize that dosage will depend upon a variety of factors, including the strength of the particular compositions employed, the age, species, condition, and body weight of the animal. The size of the dose also will be determined by the route, timing and frequency of administration as well as the existence, nature, and extent of any adverse side effects that might accompany the administration of a particular composition and the desired physiological effect. It will be appreciated by one of ordinary skill in the art that various conditions or desired results, may require prolonged treatment involving multiple administrations.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached.

The amount of the compound of the invention administered per dose or the total amount administered per day may be predetermined or it may be determined on an individual patient basis by taking into consideration numerous factors, including the nature and severity of the patient's condition, the condition being treated, the age, weight, and general health of the patient, the tolerance of the patient to the compound, the route of administration, pharmacological considerations such as the activity, efficacy, pharmacokinetics and toxicology profiles of the compound and any secondary agents being administered, and the like. Patients undergoing such treatment will typically be monitored on a routine basis to determine the effectiveness of therapy. Continuous monitoring by the physician will insure that the optimal amount of the compound of the invention will be administered at any given time, as well as facilitating the determination of the duration of treatment. This is of particular value when secondary agents are also being administered, as their selection, dosage, and duration of therapy may also require adjustment. In this way, the treatment regimen and dosing schedule can be adjusted over the course of therapy so that the lowest amount of compound that exhibits the desired effectiveness is administered and, further, that administration is continued only so long as is necessary to successfully achieve the optimum effect.

As used herein, the term “administered” includes but is not limited to topical, intramuscular, intravaginal, transderma, oral or intravenous administration by liquid, capsule, tablet, or spray. Adminstration may be by injection, whether intramuscular, intravenous, intraperitoneal or by any parenteral route. Parenteral administration can be by bolus injection or by continuous infusion. Formulations for injection may be presented in unit dosage form, for example, in ampoules or in multi-dose containers with an added preservative. The compositions may take the form of suspensions, solutions or emulsions in oily or aqueous vehicles and may contain formulatory agents such as suspending, stabilizing and/or dispersing agents. Alternatively the compositions may be in powder form (e.g., lyophilized) for constitution with a suitable vehicle, for example sterile pyrogen-free water, before use. Compositions may be delivered to a subject by inhalation by any presently known suitable technique including a pressurized aerosol spray, where the dosage unit may be controlled using a valve to deliver a metered amount.

Administration by capsule and cartridges containing powder mix of the composition can be used in an inhaler or insufflator to deliver the particles to the subject. Still other routes of administration which may be used include buccal, urethral, vaginal, or rectal administration, topical administration in a cream, lotion, salve, emulsion, or other fluid or liquid composition may also be used.

In a further embodiment, the antimicrobial peptide is a retrocyclin and/or a protegrin. In a particular embodiment, retrocyclin is retrocyclin-1 and protegrin is protegrin-1.

In another embodiment, an antimicrobial composition is provided, including a therapeutically effective amount of a retrocyclin and/or a protegrin, and optionally a plant remnant.

In still another embodiment, a process for producing a retrocyclin or protegrin polypeptide is provided. The process includes integrating a plastid transformation vector as recited above into the plastid genome of a plant cell, and growing the plant cell to thereby express said retrocyclin or protegrin. The process for producing a retrocyclin or protegrin polypeptide may be carried out by at least partially purifying the retrocylin or protegrin from the plant cell.

In yet a further embodiment, there is provided a plastid genome transformed to contain a retrocyclin or protegrin polynucleotide configured so as to express retrocyclin or protegrin.

For the purposes of promoting an understanding of the principles and operation of the invention, reference will now be made to the embodiments illustrated in the drawings and specific language will be used to describe the same. It will nevertheless be understood that no limitation of the scope of the invention is thereby intended, such alterations and further modifications in the illustrated device, and such further applications of the principles of the invention as illustrated therein being contemplated as would normally occur to those skilled in the art to which the invention pertains.

### Materials and Methods

### Construction of Chloroplast Transformation Vectors

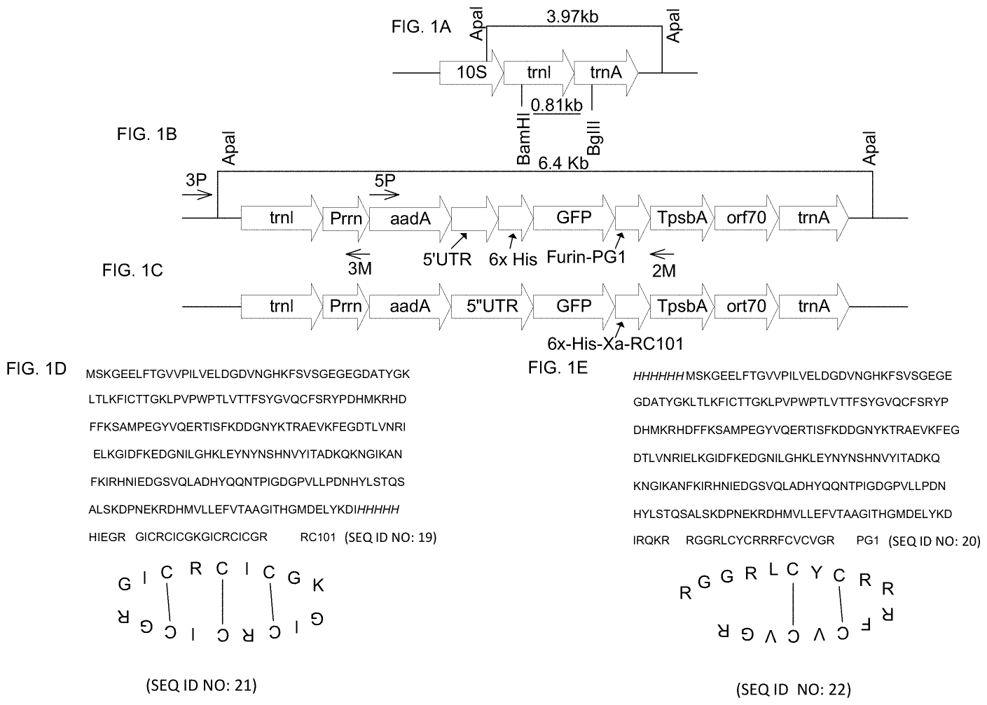

Two chloroplast transformation vectors were designed for expressing RC101 and PG1 in chloroplasts. They were constructed using the basic pLD vector, which was developed in our laboratory for chloroplast transformation (Daniell et al., Containment of herbicide resistance through genetic engineering of the chloroplast genome. Nat. Biotechnol. 16, 345-348, 1998; Verma et al., A protocol for expression of foreign genes in chloroplasts. Nat. Protoc. 3, 739-758, 2008). Both PG1 and RC101 were fused with GFP gene because of their small sizes (18 amino acids). Besides, GFP was used as a reporter and to aid in quantification of the fusion proteins. A 6-histidine tag was also engineered upstream of RC101/PG1 to facilitate purification of these fusion proteins. A furin protease cleavage site was engineered between PG1 and GFP while a Factor Xa protease cleavage site was engineered between RC-101 and the 6-histidine tag to facilitate release of PG1/RC-101 from these fusion proteins. The promoter and 5′-untranslated region (UTR) of the tobacco psbA gene was placed upstream of the His₆-GFP-Furin-PG1/GFP-His₆-Xa-RC101 transgene cassette to enhance expression of these fusion proteins. The aadA gene, which conferred resistance to spectinomycin, was driven by the constitutive Prrn promoter. The flanking sequences of trnI and trnA facilitated recombination with the native chloroplast genome (FIG. 1b-c). The transgene fragment sequences and the disulfide bonds of RC101 and PG1 are shown in FIG. 1d-e.

### Confirmation of Transgene Cassette Integration and Homoplasmy

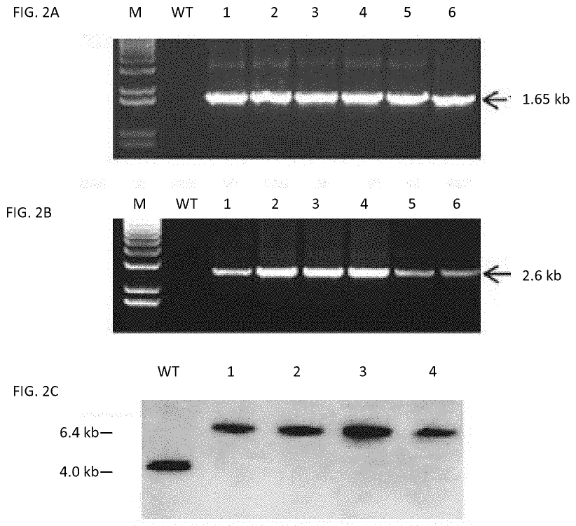

Several primary shoots appeared from the RC101 and PG1 bombarded tobacco leaves and they were developed through three rounds of selection. To confirm integration of transgene cassettes into the chloroplast genome, the putative transformed shoots were screened by PCR. Two pairs of primers were used for screening. The 3P and 3M primers were used to check site-specific integration of the selectable marker gene (aadA) into the chloroplast genome. The 5P and 2M primers were used to check integration of the transgene expression cassette (FIG. 1b-c). DNA template from the RC101-GFP and PG1-GFP transplastomic shoots yielded PCR products with both primers (FIG. 2a-b). The 3P-3M PCR products for both the RC101 and PG1 transformants were 1.65 kbp and 5P-2M PCR products were 2.6 kbp. Because the sizes of the RC101 and PG1 transgene expression cassette (including GFP) were of similar size, PCR product sizes were also similar. These PCR products could be generated only from transformed chloroplasts and not nuclear transformants or spontaneous mutants.

Because there are thousands of copies of chloroplast genomes in each plant cell, some of them may not be transformed. Therefore, Southern blot was performed to investigate whether RC101 and PG1 transplastomic plants achieved homoplasmy. The probe used was made by digesting the flanking sequences trnI and trnA with BamHI and BglII (FIG. 1a). Flanking sequence probe identified a single 4.0 kbp fragment in the untransformed tobacco, as expected. In the RC-101 and PG1 transplastomic lines, only one 6.4 kbp fragment was observed (FIG. 2c). Absence of the 4.0 kbp fragment confirmed that all the chloroplast genomes were transformed (to the detection limit of Southern blots) and therefore they are considered to be homoplasmic.

### Evaluation of RC101 or PG1 Expression in Transgenic Chloroplasts

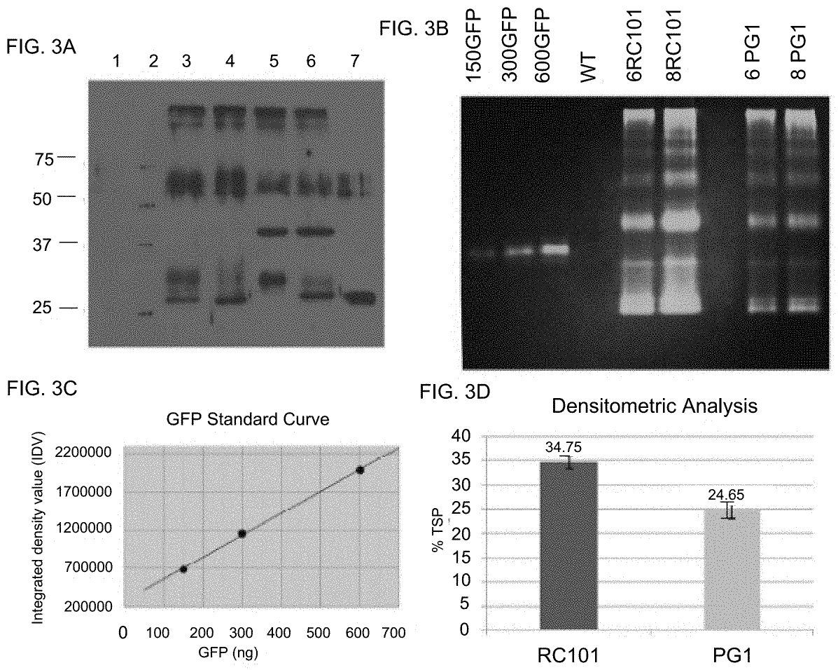

To evaluate expression of foreign genes in chloroplasts of RC101-GFP and PG1-GFP transplastomic lines, immunoblots using GFP antibodies were performed. Based on the TSP concentration, same amount of protein extracts from RC-101 and PG1 transplastomic lines (before and after protease digestion) were resolved on 12% SDS-PAGE gels. The size of RC-101 is 1.9 kDa while the size of PG1 is 2.1 kDa. Therefore, the sizes of RC101-GFP and PG1-GFP are both ˜29 kDa. After cleavage of RC101 and PG1 from GFP, we should observe only the 27 kDa GFP polypeptide. The immunoblot result is shown in FIG. 3a. Clearly, the fusion proteins were cleaved after protease digestion.

An alternative approach to confirm the expression of RC101-GFP and PG1-GFP proteins is to observe the green fluorescence emitted by GFP. After crude protein extracts were resolved on the native polyacrylamide gel, the green fluorescence emitted by GFP fusion proteins was observed under the UV light. The green peptides shown correspond to the GFP fusion proteins. The strong green fluorescence observed indicated that GFP fusion proteins were expressed at high levels (FIG. 3b). The expression of RC101 and PG1 transplastomic plants were quantified using the GFP fluorescence by densitometric analysis. The integrated density values (IDVs) of GFP fluorescence were measured by spot densitometry. The linear GFP standard curve was established using 150-600 ng of GFP standard protein (FIG. 3c). Based on this GFP standard curve, the expression levels of RC101 and PG1 transplastomic plants were estimated to be approximately 35% and 25% of TSP (FIG. 3d). To confirm the expression levels of the transplastomic plants, ELISA was also performed to determine the quantities of RC101-GFP and PG1-GFP fusion proteins in transplastomic tobacco plants. Because the antimicrobial peptides RC-101 and PG1 were fused with GFP proteins, ELISA was performed using the GFP antibodies to quantify the RC101-GFP and PG1-GFP fusion proteins. RC101-GFP accumulated to 32-38% of TSP and PG1-GFP accumulated to 17-26% of TSP. This variation of expression levels could be due to leaf samples harvested from plants under different periods of illumination.

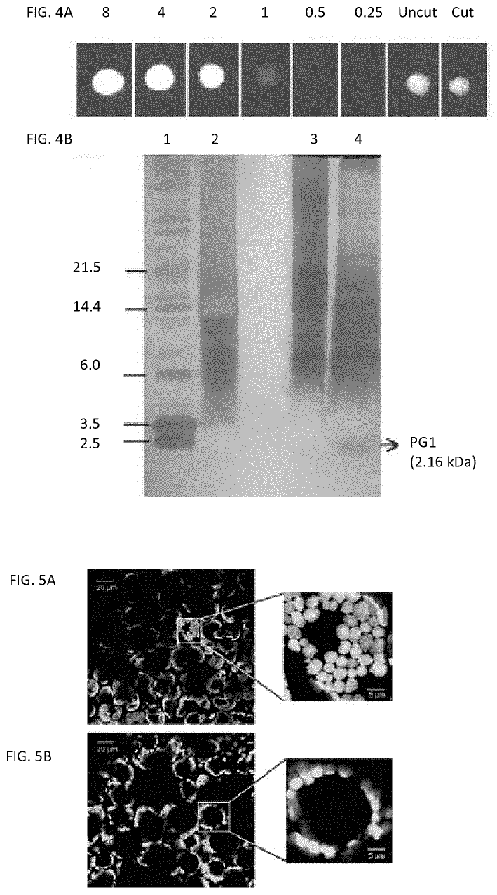

Dot blot analysis was also performed to evaluate the expression of RC-101 in transgenic chloroplasts. Factor Xa cleaved samples and uncleaved samples from RC-101 transplastomic plants were tested by dot blots. It is shown that both uncut and cut samples of RC101-GFP appeared positive (FIG. 4a). As shown in previous experiments (FIG. 3a), RC101-GFP fusion proteins were already partially cleaved by Factor Xa within chloroplasts. Because PG1 was not immunogenic, dot blot analysis could not be done with PG1 transplastomic plants. Instead, PG1 protein expression was examined by silver staining. By comparison of cut and uncut samples from PG1 transplastomic plants, it is clear that there is a 2 kDa polypeptide present in the furin digested sample but absent in the uncut sample and untransformed tobacco protein extract (FIG. 4b). The size of PG1 is 2.16 kDa and therefore this fragment should correspond to the PG1 protein.

RC101 and PG1 were Expressed and Contained within Chloroplasts

In order to investigate whether the chloroplasts remained intact when RC101 or PG1 antimicrobial peptides were highly expressed in chloroplasts, fresh leaves were examined under the confocal microscope. Strong green fluorescence was emitted from the RC-101 and PG1 transplastomic lines (FIG. 5a-b). We observed that chloroplasts emitting green fluorescence formed circles around each cell. There was no GFP fluorescence outside chloroplasts. This observation confirmed that chloroplasts remained intact because GFP fused antimicrobial proteins were not released into the cytoplasm in any detectable quantity.

### Purification of RC101-GFP and PG1-GFP Fusion Proteins

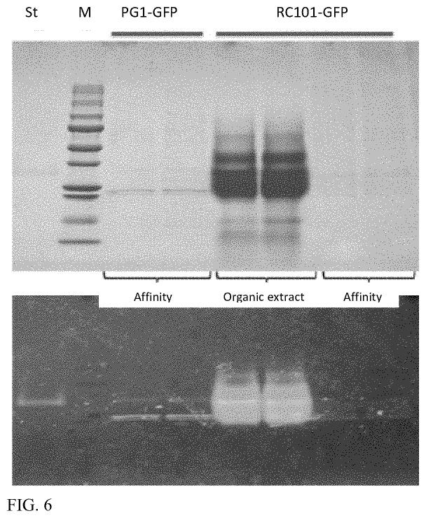

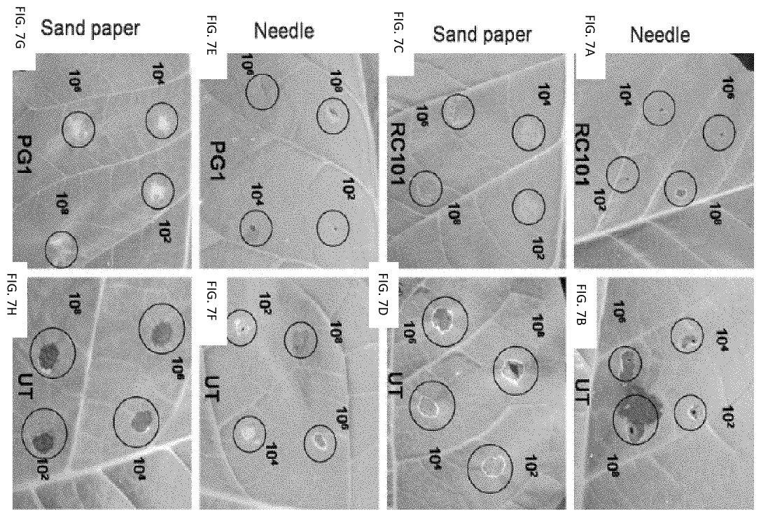

We then tried to purify the RC101-GFP and PG1-GFP fusion proteins. The engineered His-tag and GFP protein facilitated purification of RC101-GFP and PG1-GFP fusion proteins. We tried to purify the fusion proteins by affinity chromatography using His-tag or organic extraction through GFP. Results of purification using both methods are shown in FIG. 6. Approximately 8 μg of purified PG1-GFP and 5 μg of purified RC101-GFP were obtained from one gram of fresh tobacco leaf by using the affinity chromatography method. In contrast, purification of RC101-GFP using the organic extraction method resulted in a yield of 53 μg purified RC101-GFP per gram of fresh tobacco leaf. The organic extraction method resulted in much higher yield than the affinity chromatography method. It is evident that monomers, dimers and multimers of the RC101-GFP were recovered by organic extraction method, resulting in 10.6 fold higher yield whereas only the monomer was recovered using the affinity chromatography. The highly enriched fraction was the RC101-GFP monomer, ˜29 kDa in size. The upper bands should be dimers and multimers formed by RC101-GFP proteins. This same pattern was observed in the native gel electrophoresis of RC101-GFP transplastomic plant protein extracts (FIG. 3b). PG1-GFP protein was purified only by affinity chromatography, and we could observe a single band, which should be the monomer forms of PG1-GFP.

RC101 and PG1 Retained their Antimicrobial Activity when Expressed in Chloroplasts

Retrocyclin-101, as a member of the 0-defensin family, possesses antibacterial activity as well as antiviral activity (Tang et al., A Cyclic Antimicrobial Peptide Produced in Primate Leukocytes by the Ligation of Two Truncated-Defensins. Science 286, 498-502, 1999). To investigate the functionality of RC101 and PG1 expressed in the tobacco chloroplasts, we performed both antibacterial and antivirus assays using plant pathogens because use of HIV and other human bacterial pathogens require higher levels of containment than our current facilities. The antibacterial activity of RC101 and PG-1 was studied by investigating enhanced resistance to Erwinia soft rot either by using the syringe or sand paper method. One day after inoculation with Erwinia, the first signs of damage were observed on leaves of untransformed plants in the regions of inoculation. On the 3rd day, virtually all inoculated untransformed leaf surfaces underwent necrosis whereas in leaves of RC101 or PG1 transplastomic plants, no or minimally damaged zones were observed depending on the number of bacteria inoculated. Inoculation of potted plants with E. carotovora using a syringe method resulted in areas of necrosis surrounding the point of inoculation in untransformed control for all cell densities (FIG. 7b, f), whereas transplastomic RC101 and PG-1 mature leaves showed no areas of necrosis (FIG. 7a, e). Even inoculation of 10⁸ cells resulted in no or minimal necrosis in mature transplastomic leaves. In contrast, untransformed plants inoculated with 10² cells displayed obvious necrosis. Similar results were obtained with E. carotovora inoculated by the sand paper method. Transplastomic mature leaves inoculated with E. carotovora showed no necrosis (FIG. 7c) or a mild discoloration at the site of inoculation of 10⁸ cells (FIG. 7g) and untransformed plants inoculated with 10² cells or higher density displayed obvious necrosis (FIG. 7d, h).

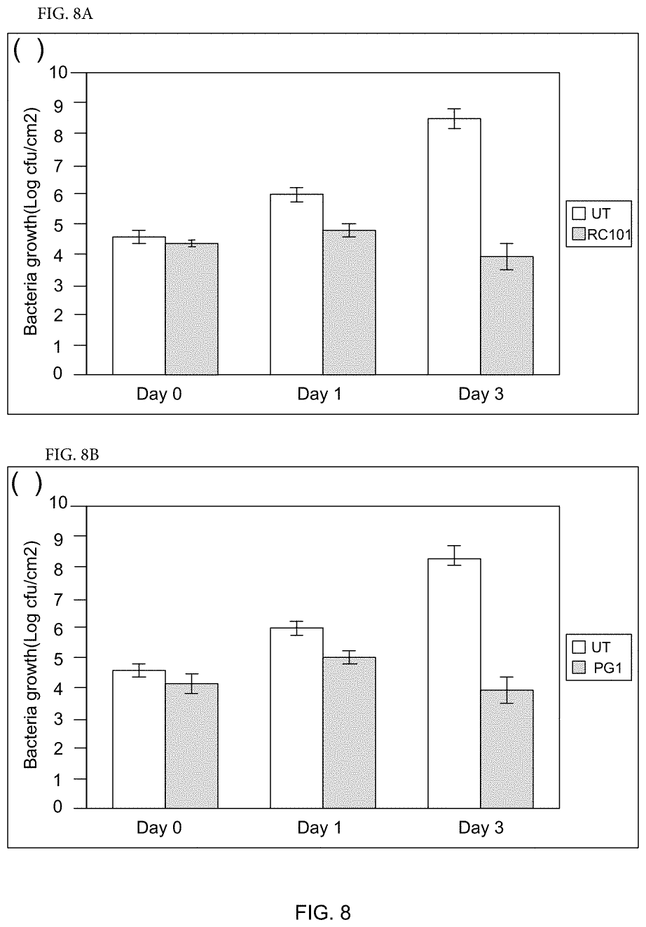

The bacterial count in inoculated plants was also estimated. Bacterial suspensions (1.0×10⁵ cfu/ml) of E. carotovora were inoculated into transplastomic and untransformed leaves by a syringe. Following inoculation, the density of E. carotovora in untransformed, RC101 and PG-1 transplastomic leaves was less than 1×10⁵ cfu/cm² at 0 day post-inoculation. Three days after inoculation, the population of E. carotovora in untransformed tobacco leaves reached 2.0×10⁸ cfu/cm² (FIG. 8a, b). In comparison, the density of E. carotovora was less than 1×10⁴ cfu/cm² in both RC101 (FIG. 8a) and PG1 (FIG. 8b) transplastomic leaves three days after inoculation, a 10-000 fold reduction in bacterial burden. In addition, no apparent symptoms of necrosis were observed in any of the RC101 or PG1 plants. These results demonstrated that the RC101 and PG1 transplastomic plants are resistant to E. carotovora. Therefore, RC101 and PG1 maintained their antibacterial activity when expressed in chloroplasts.

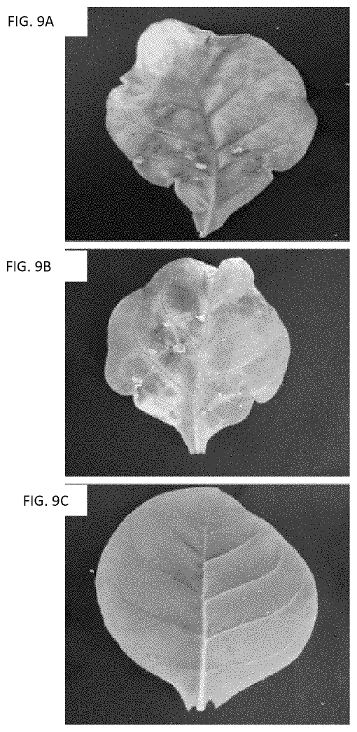

To determine the antiviral activity of PG1 and RC101 when expressed in tobacco chloroplasts, transplastomic and untransformed control plants were tested for tobacco mosaic virus (TMV) infection for 20 days. In susceptible untransformed control and PG1 plants, TMV multiplied and spread throughout the plants, causing typical mosaic, necrosis and wrinkle symptoms within 20 days after inoculation (FIG. 9a, b). However, the RC101 transplastomic plants didn't show obvious symptoms of TMV infection, and the plants grew well (FIG. 9c). These results confirmed the antiviral activity of RC101 by conferring resistance to TMV when expressed in chloroplasts.

RC101 and PG1 are antimicrobial peptides that have potent antimicrobial activities against a broad spectrum of microorganisms. Both RC101 and PG1 are disulfide-bonded proteins. RC101 contains three and PG1 contains two intramolecular disulfides bonds that are important for their antimicrobial activities (Chen et al., Development of protegrins for the treatment and prevention of oral mucositis: structure-activity relationships of synthetic protegrin analogues. Biopolymers. 55, 88-98, 2000; Harwig et al., Intramolecular disulfide bonds enhance the antimicrobial and lytic activities of protegrins at physiological sodium chloride concentrations. Eur. J Biochem. 240, 352-357 1996; Jenssen et al., Peptide Antimicrobial Agents. Clin. Microbiol. Rev. 19, 491-511, 2006; Trabi et al., Three-Dimensional Structure of RTD-1, a Cyclic Antimicrobial Defensin from Rhesus Macaque Leukocytes. Biochemistry 40, 4211-4221, 2001). Because RC101 and PG1 are microbicidal and contain multiple disulfide bonds, they have not yet been produced in microbial or cell culture systems. The present disclosure presents the discovery of how to produce low cost and functional RC101 and PG1 antimicrobial peptides in transgenic tobacco chloroplasts.

The antimicrobial peptide MSI-99 has been expressed in transgenic tobacco chloroplasts without harmful effects to transplastomic plants. MSI-99 is an analog of a naturally occurring peptide (magainin 2) found in the skin of the African frog (Jacob and Zasloff, M., Potential therapeutic applications of magainins and other antimicrobial agents of animal origin. Ciba Found. Symp. 186, 197-216, 1994). In another study, a proteinaceous antibiotic called PlyGBS lysine was expressed in tobacco chloroplasts to high levels (>70% TSP, Oey et al., Exhaustion of the chloroplast protein synthesis capacity by massive expression of a highly stable protein antibiotic. Plant J. 57, 436-445, 2009). The PlyGBS transplastomic plants showed delayed growth and a slightly pale-green phenotype when compared to the untransformed plants. The authors suggested that it was due to the exhaustion of protein synthesis capacity of transgenic chloroplasts by the massive over-expression of PlyGBS although expression of >70% TSP of CTB-proinsulin yielded healthy transplastomic plants (Ruhlman et al., 2010). Previously expressed antimicrobial peptides did not contain disulfide bonds whereas the RC101 and PG1 antimicrobial peptides have β-sheet structures and contain multiple intramolecular disulfide bonds. Therefore, efforts to express RC101 and PG1 in transgenic chloroplasts will further expand the applications of the chloroplast transformation system.

In order to facilitate expression of small antimicrobial peptides RC101 and PG1 in tobacco chloroplasts, each peptide was translationally fused with the GFP. This also facilitated detection and quantification of RC101-GFP and PG1-GFP in chloroplasts. The expression of GFP fusion proteins was visualized by examination under UV light or in immunoblots using the anti-GFP antibody. ELISA was also performed using anti-GFP antibody to quantify the expression of fusion proteins. Factor Xa protease cleavage site was inserted between RC101 and GFP and the furin cleavage site was inserted between PG1 and GFP so that they could be cleaved from their fusion proteins by appropriate proteases. The RC101-GFP protein was already partially cleaved within chloroplasts, suggesting the presence of Factor Xa like protease activity within chloroplasts.

The smaller green fluorescent peptides observed in RC101 and PG1 lanes in FIG. 3b should be the monomer forms of RC101-GFP or PG1-GFP. The monomers ran faster than the GFP standard, as GFP when fused with RC-101 or PG1, has higher electrophoretic mobility in native gels. Different sizes correspond to the multimers formed by the GFP fusion proteins. GFP protein did not form multimers. Therefore, the formation of multimers by RC101-GFP or PG1-GFP fusion proteins is most likely due to folded antimicrobial peptides RC-101 or PG1, which are both disulfide-bonded proteins. Similar folding pattern has also been observed before, when proteins containing multiple disulfide bonds were expressed in chloroplasts, including CTB-proinsulin (Ruhlman et al., Expression of cholera toxin B-proinsulin fusion protein in lettuce and tobacco chloroplasts—oral administration protects against development of insulitis in non-obese diabetic mice. Plant Biotechnol. J. 5, 495-510, 2007) and interferon-α2b (Arlen et al., Field production and functional evaluation of chloroplast-derived interferon-alpha2b. Plant Biotechnol. J. 5, 511-525, 2007).

The toxicity of antimicrobial peptides is specific against microbial membranes and therefore can be safely applied to mammals, including human beings. The composition of the membranes is likely to be the determining factor for their selectivity. Biomembranes of prokaryotic or eukaryotic cells differ significantly. Mammalian cytoplasmic membranes are mainly composed of phosphatidylcholine (PC), phosphatidylethanolamine (PE), sphingomyelin (Sph) and cholesterol, which are all generally neutrally charged. In contrast, in many bacterial pathogens, the membranes are composed predominantly of phosphatidylglycerol (PG), cardiolipin (CL) and phosphatidylserine (PS), which are highly electronegative (Yeaman and Yount, Mechanisms of Antimicrobial Peptide Action and Resistance. Pharmacol Rev. 55, 27-55, 2003). Most antimicrobial peptides, including RC101 and PG1, are positively charged under physiological pH because they are rich in Arginine. Therefore, the net negative charge of the biomembranes makes them the preferred target sites of antimicrobial peptides. The chloroplast envelope and thylakoid membranes predominantly possess three glycolipids: monogalactosyl diacylglycerol (MGDG), digalactosyl diacylglycerol (DGDG) and sulfoquinovosyl diacylglycerol (SQDG), and a sole phospholipid: phosphatidylglycerol (PG). SQDG and PG, distinct from the non-charged MGDG and DGDG, are negatively charged. However, MGDG makes up 50% of chloroplast membrane lipid and DGDG makes up 30%, suggesting that the major components of chloroplast membranes are neutral. Leaves of RC101 and PG1 transplastomic plants were examined under confocal microscope. Confocal images showed that GFP fusion proteins were contained within chloroplasts and were not released into the cytoplasm. Cationic antimicrobial peptides including RC101 and PG1 kill bacteria by disrupting their membranes. Although the chloroplast membrane structure can not be resolved from the confocal images shown in FIG. 5, no GFP fluorescence was detected outside the chloroplasts, suggesting that chloroplasts are not disrupted.

RC101-GFP and PG1-GFP accumulated up to 32-38% and 17-26% of TSP and they were purified by affinity chromatography or organic extraction method. The results showed that organic extraction resulted in nearly ten fold higher yield than the affinity chromatography method (53 μg/g vs 5 μg/g fresh leaf). PG1 was only purified by affinity chromatography and the yield was 8 μg/g fresh leaf. We did not observe dimers or multimers in RC101-GFP or PG1-GFP samples purified by affinity chromatography, which indicated that they were lost during the purification process. The His-tag was not accessible in the dimer or multimer forms of RC101-GFP and PG1-GFP. Therefore, most of the fusion proteins were not bound to the affinity column and lost during purification.

Previous studies reported that the minimum inhibitory concentrations of PG-1 against gram-positive or gram-negative bacteria ranged from 0.12 to 2 μg/ml (Steinberg et al., Protegrin-1: a broad-spectrum, rapidly microbicidal peptide with in vivo activity. Antimicrob. Agents Chemother. 41, 1738-1742, 1997). Retrocyclin (10-20 μg/ml) can inhibit proviral DNA formation and protect human CD4⁺ lymphocytes from in vitro infection by both T-tropic and M-tropic strains of HIV-1 (Cole et al., Retrocyclin: A primate peptide that protects cells from infection by T- and M-tropic strains of HIV-1. Proceedings of the National Academy of Sciences 99, 1813-1818, 2002). RC-101, as low as 2 μg, can prevent HIV-1 infection in an organ-like construct of human cervicovaginal tissue (Cole et al., The retrocyclin analogue RC-101 prevents human immunodeficiency virus type 1 infection of a model human cervicovaginal tissue construct. Immunology 121, 140-145, 2007). In another study, it was reported that Retrocyclin-1, an analogue of RC101, can kill vegetative B. anthracis cells with an minimum effective concentration <1 μg/ml (Wang et al., Retrocyclins kill bacilli and germinating spores of Bacillus anthracis and inactivate anthrax lethal toxin. J. Biol. Chem. 281, 32755-32764, 2006). As can be seen from these published data, antimicrobial peptides are highly potent and their effective dosage is only few μg/ml. Although our purification yield is relatively low, tobacco can be scaled up to yield up to 40 metric tons of biomass/acre/year. One acre of RC101 transplastomic tobacco plants could potentially yield up to 2 kg purified RC101 by organic extraction. Therefore, adequate quantities of RC101 or PG1 could be purified from transplastomic plants for preclinical or clinical studies.

RC101 and PG1 are shown to be functional when expressed in chloroplasts. Both RC101 and PG1 protected the transgenic tobacco plants from bacterial infection caused by Erwinia carotovora. In the antiviral assays, RC101 transgenic plants were resistant to TMV infection, but PG1 transgenic plants showed the symptoms of mosaic, necrosis and wrinkle as untransformed plants. Although PG1 has a broad-spectrum antimicrobial activity against bacteria, virus and fungus, it is most effective against bacterial infections, especially antibiotic-resistant bacteria (Kokryakov et al., Protegrins: leukocyte antimicrobial peptides that combine features of corticostatic defensins and tachyplesins. FEBS Letters 327, 231-2361993; Qu et al., Susceptibility of Neisseria gonorrhoeae to protegrins. Infect. Immun. 64, 1240-1245, 1996; Steinberg et al., Protegrin-1: a broad-spectrum, rapidly microbicidal peptide with in vivo activity. Antimicrob. Agents Chemother. 41, 1738-1742 1997; Yasin et al., Susceptibility of Chlamydia trachomatis to protegrins and defensins. Infect. Immun. 64, 709-713, 1996). In our study, PG1 is not effective in protecting plants from TMV infection. RC101 is an analog of retrocyclin and it is especially effective in protecting against viral infections. Several previous studies have shown that RC101 can be used to prevent HIV-1 infection (Cole et al., Retrocyclin: A primate peptide that protects cells from infection by T- and M-tropic strains of HIV-1. Proceedings of the National Academy of Sciences 99, 1813-1818, 2002; Cole et al., The retrocyclin analogue RC-101 prevents human immunodeficiency virus type 1 infection of a model human cervicovaginal tissue construct. Immunology 121, 140-145, 2007). Our study shows that RC101 is active against the retrovirus TMV when expressed in chloroplasts.

The antimicrobial activities of RC101 and PG1 can protect plants from phytopathogen infections, which make them good candidates to engineer disease resistant plants. Because the use of HIV and other human bacterial or viral pathogens require higher levels of containment than our current facilities, these studies were not performed. Future studies will include testing RC101 and PG1 in suitable animal models against bacterial or viral pathogens.

### Experimental Procedures

### Construction of Chloroplast Transformation Vectors

The 6×His-Factor Xa-RC101 sequence was synthesized by Klenow fragment and it was flanked by EcoRV and NotI restriction sites. The oligomers used were:

C2Fwd (5′-
GATATCCATCATCATCATCATCATATCGAAGGCCGCGGTATTTGTAGATG
TATTTGTGGTAAAGGTATTT-3′, SEQ ID NO: 1)
and
C2Rev (3′-
CGGCGCCATAAACATCTACATAAAC
ACCATTTCCATAAACATCTACATAAACACCATCTATTCGCCGGCG-5′,
SEQ ID NO: 2,
or
5′-
GCGGCCGCTTATCTACCACAAATACATCTACAAATACCTTTACCACAAAT
ACATCTACAAATACCGCGGC-3′, SEQ ID NO: 3).

Soluble modified GFP (sm-GFP) protein was cloned into the pGEM-T vector. The 6×His-Factor Xa-RC101 sequence was cleaved by EcoRV and NotI and subcloned into the pGEM-GFP vector. Then GFP-6×His-Factor Xa-RC101 was digested by NdeI (partial) and NotI and subcloned into the pLD vector (Daniell et al., 1998; Daniell et al., 2001).

The EcoRV-Furin-PG1-NotI sequence was synthesized by Klenow fragment. The oligomers used were: EcoRV-Start Codon-Furin-PG1 (5′-GTC-GATATC-ATG-GGCCAAAAACGAAGGGGAGGTCGCCTGTGCTATTGTAGGCGTAGGTTCTGCGT CTGT, SEQ ID NO: 4) and NotI-stop codon-reverse PG1 (5′-GCA-GCGGCCGC-TCA-TCCTCGTCCGACACAGACGCAGAACCTACGCCTACAATAGCACAGGCGACCTC CCCT-3′, SEQ ID NO: 5). A 6×His (SEQ ID NO: 6) tag was introduced by PCR to the 5′ end of smGFP protein and they were cloned into the pGEM-T vector. The synthesized Furin-PG1 gene sequence was digested by EcoRV and NotI and then inserted into the 3′ end of 6×His-smGFP sequence in the pGEM-T vector. The 6×His-smGFP-Furin-PG1 sequence was digested by the NdeI (partial) and NotI enzymes and subcloned into the pLD vector.

### Bombardment and Selection of Transplastomic Plants

Sterile tobacco leaves were bombarded using the Bio-rad PDS 1000/He biolistic device as described previously (Verma et al., 2008). Bombarded leaves were then subjected to three rounds of selection. First two rounds of selection were performed on the regeneration medium of plants (RMOP) and the third round of selection was on Murashige and Skoog medium without hormones (MSO) medium. All these were supplemented with 500 mg/L spectinomycin. After selection, RC-101 and PG1 transplastomic shoots were transferred to pots in the greenhouse.

### PCR Analysis to Confirm Transplastomic Plants

Total plant DNA was isolated from transplastomic tobacco leaves using the DNeasy Plant Mini Kit from Qiagen. PCR was set up with two pairs of primers, 3P-3M and 5P-2M (Verma et al., 2008) to confirm the successful transformation of tobacco chloroplasts. The 3P primer (AAAACCCGTCCTCAGTTCGGATTGC, SEQ ID NO: 7) anneals with the native chloroplast genome and 3M primer (CCGCGTTGTTTCATCAAGCCTTACG, SEQ ID NO: 8) anneals with the aadA gene. Therefore this pair of primers was used to check site-specific integration of selectable marker genes into the chloroplast genome. The 5P primer (CTGTAGAAGTCACCATTGTTGTGC, SEQ ID NO: 9) anneals with the aadA gene and 2M primer (TGACTGCCCACCTGAGAGCGGACA, SEQ ID NO: 10) anneals with the trnA gene, which were used to check integration of the transgene expression cassette.

### Southern Blot to Confirm Homoplasmy

Total plant DNA was digested with ApaI enzyme and then separated on a 0.8% agarose gel. After electrophoresis, the gel was soaked in 0.25N HCl depurination solution for 15 minutes, and then rinsed twice in water, 5 minutes each. After that, the gel was soaked in transfer buffer (0.4N NaOH, 1M NaCl) for 20 minutes, and then the dry transfer was set up. After transfer, the membrane was rinsed with 2×SSC twice for 5 minutes each. After the membrane was dry, it was cross-linked using GS GeneLinker UV Chamber at C3 setting. The 0.81 kbp flanking sequence probe was prepared by digesting pUC-CT vector with BamHI and BglII (FIG. 1a). After the probe was labeled with ³²P, hybridization of the membrane was done by using Stratagene QUICK-HYB hybridization solution and protocol (Stratagene, La Jolla, Calif.).

### Factor Xa and Furin Cleavage Assays

RC-101 tobacco transplastomic leaves (100 mg) were ground in liquid nitrogen and homogenized in 200 μl of plant extraction buffer (0.1 N NaOH, 1 M Tris-HCl, pH4.5) using a mechanical mixer. The homogenized plant extract was then centrifuged for 5 minutes at 14,000 rpm at 4° C. The extract (10 μg) was then incubated with 1 μg of Factor Xa protease in 20 mM Tris-HCl (pH 8.0 @ 25° C.) with 100 mM NaCl and 2 mM CaCl₂ overnight at 23° C. The cleaved products were loaded with uncleaved RC-101 protein extracts on the same gel to investigate cleavage of RC101-GFP fusion protein. Western blot analysis was performed as described below.

Total protein from the PG1-GFP transplastomic tobacco leaves were extracted the same way as RC101-GFP described above. The extract (10 μg) from PG1-GFP transplastomic tobacco leaves was incubated with 1 unit of furin in a total reaction volume of 25 μl containing 100 mM Hepes (pH7.5, 25° C.), 0.5% Triton X-100, 1 mM CaCl2, 1 mM 2-mercaptoethanol at 25° C.

### Native Polyacrylamide Gel Electrophoresis and Densitometric Analysis

Total protein from the RC101-GFP and PG1-GFP transplastomic plants were extracted as described above. The TSP concentration was determined by the Bradford assay and then different amount of TSP was loaded with native gel loading buffer (60 mM pH 6.8 Tris-HCl, 25% glycerol and 0.01% Bromophenol blue) into the 12% native polyacrylamide gel. After electrophoresis, the gel was scanned and analyzed for the presence of GFP fusion proteins using AlphaImager® and AlphaEase® FC software (Alpha Innotech, San Leandro, Calif., USA). The integrated density values (IDVs) of the GFP standards and samples were recorded and analyzed further.

### Western Blot Analysis

Frozen leaf materials (100 mg) were ground in liquid nitrogen and then resuspended in 200 μl of plant extraction buffer. The supernatant was collected after centrifuging the sample for 5 minutes at 14,000 rpm. The plant extract was mixed with 2× sample loading buffer and then boiled for 5 minutes before loading. The transformed, untransformed plant extracts and recombinant GFP standard (Vector Labs) were loaded onto the 12% SDS-PAGE gel. The proteins in the gel were then transferred to the nitrocellulose membrane at 100V for 1 hour. After transfer, the membrane was first blocked in PTM (1×PBS, 0.1% Tween-20, 3% milk) for 2 hours at room temperature and then incubated with chick anti-GFP primary antibody (Chemicon) at 1:3000 dilution in PTM for 2 hours at room temperature. After the membrane was washed 3 times with PBS-T (1×PBS, 0.1% Tween-20), 5 minutes each time, rabbit anti-chick secondary antibody conjugated with HRP was added at 1:3000 dilution in PTM and then incubated for 1 hour at room temperature.

### Dot Blot Assay

The Immobilon-P (PVDF) membrane was pre-wet in methanol for 1-2 min, rinsed twice with TBS (500 mM NaCl, 20 mM Tris-HCl pH 7.5) and the membrane was soaked in TBS until use. Protein extracts from RC101 transplastomic line and standards (0.25-8 ng of RC101 peptides) were resuspended in 0.1% acetic acid and then dotted onto an Immobilon-P membrane. Once the last dot was soaked in, the membrane was placed in fixation buffer (0.05% glutaraldehyde in 1×TBS) and rocked on the orbital shaker at room temperature for 20 min. The membrane was blocked for 30 min at 37° C. using Superblock (Pierce, Rockford, Ill.) and then incubated overnight with anti-RC101 polyclonal antisera (Invitrogen custom antibody service, Carlsbad, Calif.) diluted 1:2000 in antibody buffer (Superblock diluted 1:3 in TBS containing 0.05% Tween-20 and 0.01% thimerosal). After washing twice and blocked again for 15 min, the membrane was incubated with peroxidase-conjugated anti-rabbit immunoglobulin G for 1 hr. After washing, the membrane was developed with Immun-Star HRP (Bio-Rad, Hercules, Calif.). Images were captured and analyzed using the Bio-Rad ChemiDoc system.

### PG-1 Furin Cleavage Assay and Silver Staining

After furin digestion, PG1 was cleaved off from GFP. Because of non-availability of PG1 antibody, we used silver staining to investigate the presence of the 2.1 kDa PG1 protein after furin cleavage. The cleaved products of PG1-GFP fusion protein were separated in a 16.8% tris-tricine gel to get the maximum resolution in the <10 kDa range. Untransformed plant extracts, Marker 12 unstained standard (Invitrogen), PG1-GFP plant protein extracts before and after furin digestion were mixed with sample loading buffer and loaded on the 16.8% gel. After electrophoresis, the gel was stained by silver staining.

### Confocal Microscopy

Untransformed, RC101-GFP and PG1-GFP transplastomic tobacco leaves were harvested fresh before microscopic analysis. They were cut into 5 mm×5 mm small pieces and fixed on slides. Confocal microscope (Olympus FluoView) with adjustable bandwidths of the detected fluorescence wavelength was used. The filter used was 505-525 nm. GFP fluorescence from the samples was detected and saved as digital format files.

### ELISA Quantification of RC101-GFP and PG1-GFP Fusion Proteins

All untransformed, transplastomic plant protein extracts (all the extracts used here were the same as used in Bradford assay) and recombinant GFP standard (Vector Laboratories, MB-0752) were diluted using the ELISA coating buffer (15 mM Na₂CO₃, 35 mM NaHCO₃, pH 9.6). The recombinant GFP standard was serially diluted from 100 ng/ml to 3.125 ng/ml. Different dilutions of test samples were prepared ranging from 1:1000 to 1:9000. The wells of a 96-well microtiter EIA plate were coated with 100 μl of diluted test samples and standards. The plate was covered with an adhesive plastic and incubated for 2 hours at room temperature. After incubation, the coating solution was removed and the plate was washed twice by filling the wells with 200 μl PBS and once by water. The coated wells were blocked by adding 200 μl of blocking buffer (3% dry milk in PBS). Then the plate was covered and incubated for 2 hours at room temperature. After removing the blocking buffer, the plate was washed again as described before. Mouse anti-GFP IgG monoclonal antibody (Chemicon, MAB3836) at 1:2000 dilution was added and incubated for 2 hours at room temperature. After washing twice with PBS and once with water, HRP conjugated goat anti-mouse IgG antibody (American Qualex) at 1:2000 dilution was added and incubated for 2 hours at room temperature. After washing, the plate was developed with TMB (3,3′,5, 5′-Tetramethylbenzidine). The absorbance of each well was read with a microplate reader (Biorad, model 680).

### Purification of RC101-GFP and PG1-GFP Fusion Proteins by Affinity Chromatography or Organic Extraction

Fresh leaves (10 g) were ground in liquid nitrogen. Lysis buffer (10 mM Imidazole (pH 8.0), 50 mM Na/K Phosphate buffer, 20 mM Tris-HCl, 300 mM NaCl) (75-80 ml) and one tablet of protease inhibitor cocktail (Roche-Complete, EDTA-free) was added to the ground leaf powder. The sonicated sample was centrifuged at 75000 g for 1 hr at 10° C. The supernatant was filtered using a Mira cloth to remove debris and loaded onto the column.

The sample lines of the AKTA-3D FPLC were primed and purged before loading the samples at the rate of 3 ml/min. The fraction size was 2.5 ml. The samples were subjected to affinity chromatography and the elution was done at 100% gradient which was 250 mM imidazole. The peak at the right wavelength (498 nm) was noted and all the fractions comprising that peak were taken. The purified proteins were then separated on the native PAGE gel. The gel was stained by coomassie staining and viewed directly.

The organic extraction protocol described by Skosyrev et al. (Skosyrev et al., 2003) was used. Saturated ammonium sulfate (pH 7.8) was added to a final saturation of 70% to the plant protein extract. The entire suspension was extracted twice with a one-fourth and a 1/16th volume of ethanol by vigorous shaking for 1 min. After centrifugation, both ethanol phases were collected carefully to avoid disturbance of the interphase. A one-fourth volume of n-butanol was added to the combined ethanol extract. After vigorous shaking and centrifugation, the lower phase containing fusion protein was carefully collected. Lower phase was adjusted to 20% saturation of ammonium sulfate and loaded directly to a column with Butyl-Toyopearl equilibrated with 20% ammonium sulfate in 10 mM Tris-HCl, pH 7.8. After washing with the equilibration buffer, protein was eluted with salt-free 10 mM Tris-HCl, pH 7.8.

### In Planta Assay for Resistance to Erwinia Soft Rot

To investigate bacterial resistance of RC101 and PG-1 transplastomic line, untransformed control and transplastomic leaves were inoculated with bacterial suspension culture. Erwinia carotovora strain was obtained from Dr. Jerry Bartz's laboratory (University of Florida, Gainesville) and grown for 24 h at 25° C. in 5 ml of Nutrient broth (NB) medium (Difco). Different dilutions of bacteria were prepared. Five- to 7-mm areas of green house grown untransformed, RC101 and PG-1 transplastomic tobacco leaves were scraped with fine-grain sandpaper and 20 μl of 10⁸, 10⁶, 10⁴ and 10² of Erwinia cells were inoculated to each prepared area. In a parallel study, 20 μl of 10⁸, 10⁶, 10⁴ and 10² of Erwinia cells were injected into leaves of untransformed, RC101 and PG-1 transplastomic tobacco using a syringe with a precision glide needle. Photos were taken 5 days after inoculation.

Erwinia carotovora Inoculation and Analysis

The leaves of untransformed and transplastomic tobacco plants were inoculated with 20 μl of bacterial suspension (1.0×10⁵ cfu/ml) through a syringe. Each leaf disc (0.8 cm diameter) was punched off from the inoculated area of an individual plant after 0, 1 or 3 days of incubation. The bacterial population inside the leaf was calculated as follows. Leaf tissue was ground in 100 μl sterilized water in a microcentrifuge tube. The suspension was serially diluted with sterilized water and was then plated on nutrient broth agar plates (Difco). Colonies were counted after one day of incubation at 25° C.

### Tobacco Mosaic Virus (TMV) Inoculation and Analysis

Full-length infectious TMV RNA transcripts were generated by in vitro transcription of KpnI-linearized Klenow-filled pTMV004 vector (Obtained from Prof. William Dawson, University of Florida Citrus Research and Education Center, Lake Alfred) using T7 RNA polymerase (Promega), as described before (Dinesh-Kumar and Baker, 2000). In vitro generated TMV transcripts were rub-inoculated onto tobacco plants and infected leaves were harvested 14 days after inoculation and re-inoculated onto tobacco plants for virus multiplication. The inoculum for plant infection was prepared by grinding infected TMV-sensitive tobacco leaf tissues in 10 mM sodium phosphate buffer, pH 7.0. The leaf sap with virus was then injected into the main veins of 4- to 5-week-old PG1, RC101 transplastomic and untransformed tobacco plant leaves using a syringe. Plants were evaluated for development of symptoms to TMV infection for 20 days after inoculation.

Provided below are examples of retrocylin and protegrin sequences.

| R1R1 or A1A1 | R1R3 or A1A3 | R3R3 or A3A3 |
|---|---|---|
| Homodimer | Heterodimer | Homodimer |
| GICRCICGRGICRCICGR | GICRCICGKGICRC | GICRCICGKGIC |
| (SEQ ID NO: 11) | ICGR | RCICGK |
| | (SEQ ID NO: 12) | (SEQ ID |
| | | NO: 13) |
| Protegrin-1 | Protegrin-4 | |
| RGGRLCYCRRRFCVCVGR | RGGRLCYCRGWICF | |
| | CVGR | |
| (SEQ ID NO: 14) | (SEQ ID NO: 17) | |
| Protegrin-2 | Protegrin-5 | |
| RGGRLCYCRRRFCVCV | RGGRLCYCRPRFCV | |
| | CVGR | |
| (SEQ ID NO: 15) | (SEQ ID NO: 18) | |
| Protegrin-3 | | |
| RGGGLCYCRRRFCVCVGR | | |
| (SEQ ID NO: 16) | | |

In each of SEQ ID NOs: 11, 12 and 13, the C at the 3^{rd} position is disulfide bonded to the C in the 16^{th} position;
In each of SEQ ID NOs: 11, 12 and 13, the C at the 5^{th} position is disulfide bonded to the C in the 14^{th} position;
In each of SEQ ID NOs: 11, 12 and 13, the C at the 7^{th} position is disulfide bonded to the C in the 12^{th} position.
Peptide sequences of exemplary retrocyclin peptides in dimer structure form (SEQ ID NOs: 11, 12 and 13) for use in accordance with the teachings herein. The dimer or monomer (SEQ ID NOs: 23 and 24) nonapeptides may be used.
In each of SEQ ID NOs: 14, 15, 16, 17 and 18, the C at the 6^{th} position is disulfide bonded to the C in the 15^{th} position;
In each of SEQ ID NOs: 14, 15, 16, 17 and 18, the C at the 8^{th} position is disulfide bonded to the C in the 13^{th} position.
Peptide sequences and structures of protegrin molecules for use in accordance with the teachings herein.

It should be borne in mind that all patents, patent applications, patent publications, technical publications, scientific publications, and other references referenced herein are hereby incorporated by reference in this application in order to more fully describe the state of the art to which the present invention pertains.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skill in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

While one or more embodiments of the present invention have been shown and described herein, such embodiments are provided by way of example only. Variations, changes and substitutions may be made without departing from the invention herein. Accordingly, it is intended that the invention be limited only by the spirit and scope of the appended claims. The teachings of all references cited herein are incorporated in their entirety to the extent not inconsistent with the teachings herein.

1.-19. (canceled)

20. A method of treating, or delaying the onset of a viral or bacterial infection, comprising administering to a subject a composition comprising a therapeutically effective amount of at least one antimicrobial peptide expressed in and obtained from a chloroplast in a transplastomic plant wherein said peptide is selected from the group consisting of RC-100, RC-101, RC-101-2K, each of said RC-100, RC-101, RC-101-2K being in dimer or monomer form, protegrin 1, protegrin 2, protegrin 3, protegrin 4, and protegrin 5, said at least one antimicrobial peptide retaining antimicrobial and, or anti-viral activity following purification from said chloroplast wherein said transplastomic plant is resistant to E. carotorvora and, or TMV infection.

21. A method of treating, or delaying the onset of a viral or bacterial infection, comprising administering to a subject a composition comprising a therapeutically effective amount of two antimicrobial peptides expressed in and obtained from a chloroplast in a transplastomic plant wherein one of said peptides is selected from the group consisting of RC-100, RC-101, RC-101-2K, each of said RC-100, RC-101, RC-101-2K being in dimer or monomer form, and the other peptide is selected from protegrin 1, protegrin 2, protegrin 3, protegrin 4, and protegrin 5, said at least one antimicrobial peptide retaining antimicrobial and, or anti-viral activity following purification from said chloroplast and said transplastomic plant is resistant to E. carotorvora and TMV infection said antimicrobial peptide being expressed in the leaves of said transplastomic plant as a fusion protein comprising GFP operably linked via a furin cleavage site, wherein said antimicrobial peptides are extracted from homogenized transplastomic leaves following furin digestion, wherein said composition is administered topically, intramuscularly, intravaginally, transdermally, orally, or intravenously.

22. The method of claim 21, wherein said at least one antimicrobial peptides are RC101 and protegrin-1 and accumulate up to 32-38% and 17-26% of total soluble protein in said transplastomic plant, said method comprising assessing said composition for conferring resistance to microbial and viral infection.

23. The method of claim 20, wherein said composition is administered topically, intramuscularly, intravaginally, transdermally, orally, or intravenously.

24. An antimicrobial and antiviral composition comprising a therapeutically effective amount of retrocyclin and protegrin, expressed in, and obtained from a transplastomic plant chloroplast and a plant remnant from said transplastomic plant, said plant chloroplast being obtained from a plant resistant to E. carotovora and TMV infection.

25. A stable plastid transformation and expression vector which comprises an expression cassette comprising, as operably linked components in the 5′ to the 3′ direction of translation, a promoter operative in said plastid, a selectable marker sequence, a heterologous polynucleotide sequence coding for one or more of a polypeptide having anti-microbial activity comprising at least 95% identity to a retrocyclin protein selected from the group consisting of SEQ ID NOS: 11, 12 or 13 in monomer or dimer form or protegrin protein selected from the group consisting of SEQ ID NOS: 14, 15, 16, 17 and 18, transcription termination sequences functional in said plastid, and flanking each side of the expression cassette, flanking DNA sequences which are homologous to a DNA sequence of the target plastid genome and suitable for effecting homologous recombination of said vector into the plastid genome of the target plant, said target plant being resistant to E. carotovora and, or TMV infection upon expression of said one or more polypeptide having antimicrobial and, or antiviral activity from said plastid transformation and expression vector, wherein the selectable marker sequence is an antibiotic-free selectable marker.

26. A vector of claim 25, wherein the vector expresses a protegrin and a retrocyclin in chloroplasts of said target plant, said plant being resistant to E. carotovora and TMV infection upon expression of said protegrin and retrocyclin.

27. A stably transformed plant which comprises plastids stably transformed with the vector of claim 26 or the transgenic progeny thereof, including transgenic seeds, said plant, or transgenic progeny being resistant to E. carotovora and TMV infection.

28. A stably transformed plant of claim 9 which is a monocotyledonous or dicotyledonous plant.

29. A stably transformed plant of claim 27 which is maize, rice, grass, rye, barley, oat, wheat, soybean, peanut, grape, potato, sweet potato, pea, canola, tobacco, tomato or cotton.

30. A stably transformed plant of claim 28 which is edible for mammals and humans.

31. A stably transformed plant of claim 27 in which all the chloroplasts are uniformly transformed.

32. The composition of claim 24, wherein said plant remnant also comprises rubisco.

33. The method of claim 20, wherein said chloroplasts comprise protegrin 1 and RC101 and said method is for the treatment of sexually transmitted diseases (STDs).

34. The method of claim 21, for the treatment of sexually transmitted diseases (STDs).


SEQUENCE LISTING (24 sequences)

SEQ ID NO: 1 - DNA, 70 bp, Artificial Sequence
  Description of Artificial Sequence Synthetic oligonucleotide
gatatccatcatcatcatcatcatatcgaaggccgcggtatttgtagatgtatttgtggtaaaggtattt

SEQ ID NO: 2 - DNA, 70 bp, Artificial Sequence
  Description of Artificial Sequence Synthetic oligonucleotide
cggcgccataaacatctacataaacaccatttccataaacatctacataaacaccatctattcgccggcg

SEQ ID NO: 3 - DNA, 70 bp, Artificial Sequence
  Description of Artificial Sequence Synthetic oligonucleotide
gcggccgcttatctaccacaaatacatctacaaatacctttaccacaaatacatctacaaataccgcggc

SEQ ID NO: 4 - DNA, 69 bp, Artificial Sequence
  Description of Artificial Sequence Synthetic oligonucleotide
gtcgatatcatgggccaaaaacgaaggggaggtcgcctgtgctattgtaggcgtaggttctgcgtctgt

SEQ ID NO: 5 - DNA, 71 bp, Artificial Sequence
  Description of Artificial Sequence Synthetic oligonucleotide
gcagcggccgctcatcctcgtccgacacagacgcagaacctacgcctacaatagcacaggcgacctcccct

SEQ ID NO: 6 - protein, 6 aa, Artificial Sequence
  Description of Artificial Sequence Synthetic 6xHis tag
HHHHHH

SEQ ID NO: 7 - DNA, 25 bp, Artificial Sequence
  Description of Artificial Sequence Synthetic primer
aaaacccgtcctcagttcggattgc

SEQ ID NO: 8 - DNA, 25 bp, Artificial Sequence
  Description of Artificial Sequence Synthetic primer
ccgcgttgtttcatcaagccttacg

SEQ ID NO: 9 - DNA, 24 bp, Artificial Sequence
  Description of Artificial Sequence Synthetic primer
ctgtagaagtcaccattgttgtgc

SEQ ID NO: 10 - DNA, 24 bp, Artificial Sequence
  Description of Artificial Sequence Synthetic primer
tgactgcccacctgagagcggaca

SEQ ID NO: 11 - protein, 18 aa, Artificial Sequence
  Description of Artificial Sequence Synthetic peptide
GICRCICGRGICRCICGR

SEQ ID NO: 12 - protein, 18 aa, Artificial Sequence
  Description of Artificial Sequence Synthetic peptide
GICRCICGKGICRCICGR

SEQ ID NO: 13 - protein, 18 aa, Artificial Sequence
  Description of Artificial Sequence Synthetic peptide
GICRCICGKGICRCICGK

SEQ ID NO: 14 - protein, 18 aa, Artificial Sequence
  Description of Artificial Sequence Synthetic peptide
RGGRLCYCRRRFCVCVGR

SEQ ID NO: 15 - protein, 16 aa, Artificial Sequence
  Description of Artificial Sequence Synthetic peptide
RGGRLCYCRRRFCVCV

SEQ ID NO: 16 - protein, 18 aa, Artificial Sequence
  Description of Artificial Sequence Synthetic peptide
RGGGLCYCRRRFCVCVGR

SEQ ID NO: 17 - protein, 18 aa, Artificial Sequence
  Description of Artificial Sequence Synthetic peptide
RGGRLCYCRGWICFCVGR

SEQ ID NO: 18 - protein, 18 aa, Artificial Sequence
  Description of Artificial Sequence Synthetic peptide
RGGRLCYCRPRFCVCVGR

SEQ ID NO: 19 - protein, 268 aa, Artificial Sequence
  Description of Artificial Sequence Synthetic polypeptide
MSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKDIHHHHHHIEGRGICRCICGKGICRCICGR

SEQ ID NO: 20 - protein, 268 aa, Artificial Sequence
  Description of Artificial Sequence Synthetic polypeptide
HHHHHHMSKGEELFTGVVPILVELDGDVNGHKFSVSGEGEGDATYGKLTLKFICTTGKLPVPWPTLVTTFSYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKDIRQKRRGGRLCYCRRRFCVCVGR

SEQ ID NO: 21 - protein, 18 aa, Artificial Sequence
  Description of Artificial Sequence Synthetic peptide
GICRCICGKGICRCICGR

SEQ ID NO: 22 - protein, 18 aa, Artificial Sequence
  Description of Artificial Sequence Synthetic peptide
RGGRLCYCRRRFCVCVGR

SEQ ID NO: 23 - protein, 9 aa, Artificial Sequence
  monomer nanopeptide
GICRCICGR

SEQ ID NO: 24 - protein, 9 aa, Artificial Sequence
  monomer nanopeptide
GICRCICGK